(19) United States
(12) Patent Application Publication
(10) Pub. No.: US 2011/0230683 A1
(43) Pub. Date: Sep. 22, 2011

(54) Process and apparatus for producing ethylenically unsaturated halogenated hydrocarbons

(76) Inventors: Michael Benje, Darmstadt (DE); Peter Kammerhofer, Burgkirchen (DE); Klaus Krejci, Burghausen (DE); Rainer Kampschulte, Burghausen (DE); Helmut Grumann, Perach (DE)

(21) Appl. No.: 12/998,172

(22) PCT Filed: Sep. 3, 2009
(86) PCT No.: PCT/EP2009/006384

§ 371 Date: Jun. 6, 2011

(30) Foreign Application Priority Data
Sep. 26, 2008 (DE) 10 2008 049 260.4

(51) Int. Cl. C07C 17/25; G05D 7/00; B01J 19/00; B01J 19/08

(52) U.S. Cl. 570/226; 422/111; 422/198; 422/186.04

(57) ABSTRACT

Process and apparatus for producing ethylenically unsaturated halogenated hydrocarbons

The invention relates to a process and an apparatus for preparing ethylenically unsaturated halogenated hydrocarbons, preferably vinyl chloride by thermal dissociation of 1,2-dichloroethane, using physical or chemical measures which initiate the dissociation reaction.

The process/apparatus described makes it possible to increase the amount produced using dissociation reactors of a given size considerably. Use is made here of initiating measures to increase the heat flux through the wall of the reaction tube and at the same time the feed stream and the heating power of the reaction furnace are increased so that the conversion of the reaction is not significantly increased compared to processes without use of initiating measures.

To be able to continue to operate the process economically despite the reduction in the reaction temperature, the process parameters have to be set so that at least 50% of the amount of feed used are vaporized by means of the sensible heat content of the reaction mixture leaving the reaction zone.

### CLAIM FOR PRIORITY

This substitute specification is submitted as a national phase entry of International Patent Application No. PCT/EP2009/006384 (International Publication No. WO 2010/034397), filed Sep. 3, 2009, entitled “Method and Device for Producing Ethylenically Unsaturated Halogenated Hydrocarbons” which claims priority to German Patent Application No. DE 10 2008 049 260.4, filed Sep. 26, 2008 and is entitled, “Verfahren Und Vorrichtung Zur Herstellund Von Ethylenisch Ungesättigren Halogenierten Kohlenwasserstoffen”. The priorities of International Patent Application No. PCT/EP2009/006384 and German Patent Application No. DE 10 2008 049 260.4 are hereby claimed and their disclosures incorporated herein by reference in their entireties.

### DESCRIPTION

The present invention relates to a particularly economical process and an apparatus suitable therefor for preparing ethylenically unsaturated halogen compounds by thermal dissociation of halogenated aliphatic hydrocarbons, in particular the preparation of vinyl chloride by thermal dissociation of 1,2-dichloroethane.

The invention is described below by way of example for the production of vinyl chloride (hereinafter referred to as VCM) by thermal dissociation of 1,2-dichloroethane (hereinafter referred to as EDC), but can also be used for the preparation of other ethylenically unsaturated halogen compounds.

Terminology used herein is given its ordinary meaning unless otherwise stated herein.

### BRIEF DESCRIPTION OF THE DRAWINGS

The invention is described in detail below with reference to the drawings wherein like numbers designate similar parts and wherein:

FIG. 1 is a schematic of a reactor for producing ethylenically unsaturated halogenated hydrocarbons for halogenated aliphatic hydrocarbons as described herein.

FIG. 1A is a schematic of an older style of reactor for producing ethylenically unsaturated halogenated hydrocarbons for halogenated aliphatic hydrocarbons retrofitted to accept the invention as described herein.

FIG. 2 is a schematic of the integration of the reactor of FIG. 1 into a system for producing ethylenically unsaturated halogenated hydrocarbons for halogenated aliphatic hydrocarbons as described herein.

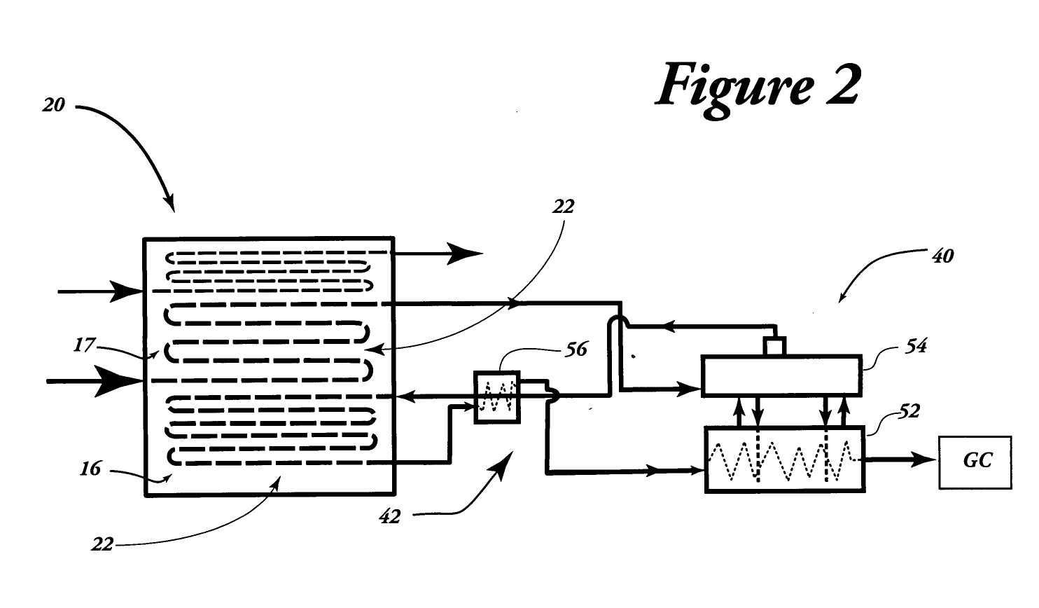

### DETAILED DESCRIPTION

VCM is nowadays prepared predominantly by thermal dissociation of EDC, with the reaction being carried out industrially according to the equation

C₂H₄Cl₂+71 kJ→C₂H₃Cl+HCl

in a reaction tube which is in turn located in a gas- or oil-heated furnace.

The reaction is usually allowed to proceed to a conversion of 55-65%, based on the EDC used (hereinafter feed EDC). The temperature of the reaction mixture leaving the furnace (hereinafter furnace exit temperature) is about 480-520° C. The reaction is carried out under superatmospheric pressure. Typical pressures at the furnace inlet are about 13-30 bar abs. in present-day processes.

At higher conversions and, resulting therefrom, a higher partial pressure of VCM in the reaction mixture, VCM is increasingly converted under the reaction conditions into subsequent products such as acetylene and benzene which in turn are precursors of carbon deposits. The formation of carbon deposits makes shutdown and cleaning of the reactor at regular intervals necessary. In view of this, a conversion of 55%, based on the EDC used, has been found to be particularly advantageous in industrial practice.

The majority of processes employed at present operate using cuboidal furnaces 20 in which the reaction tube 22 is arranged centrally as a serpentine tube 22 made up of horizontal tubes 22a, 22s, 22b arranged vertically above one another, with the serpentine tube 22 being able to have a single or double configuration. In the case of a single configuration, the tubes 22a, 22s, 22b can either be aligned or offset. The furnaces 20 are heated by means of burners 26, 28 which are arranged in rows in the furnace walls 24. The transfer of heat to the reaction tubes 22b occurs predominantly by wall and gas radiation but also convectively via the flue gas 38 formed in heating by means of burners 26. The dissociation of EDC is sometimes also carried out in other types of furnace having a different arrangement of the reaction tubes and the burners.

The invention can in principle be applied to all types of furnace 20 and burner 26, 28 arrangements and also to other ways of heating the reaction.

A typical tube reactor used for the dissociation of EDC comprises a furnace 20 and a reaction tube 22. In general, such a furnace fired by means of a primary energy carrier, e.g. oil or gas, is divided into a radiation zone 16 and a convection zone 17.

In the radiation zone 16, the heat required for the dissociation is transferred to the reaction tube 22 primarily by radiation from the burner-heated furnace walls 24 and the hot flue gas 38.

In the convection zone 17, the energy content of the hot flue gases 38 leaving the radiation zone 16 is utilized by convective heat transfer. In this way, the starting material for the dissociation reaction, e.g. EDC, can be preheated, vaporized or superheated. The generation of steam and/or the preheating of combustion air is likewise possible.

In a typical arrangement as described, for example, in EP 264,065 A1 (incorporated herein by reference in its entirety), liquid EDC is firstly preheated in the convection zone 17 of the dissociation furnace 20 and then vaporized in a specific vaporizer 40 outside the dissociation furnace 20. The gaseous EDC is then fed into the convection zone 17 again and superheated there, preferably in the shock tubes 22s, with the dissociation reaction being able to commence here. After superheating has occurred, the EDC enters the radiation zone 16 where the conversion into vinyl chloride and hydrogen chloride takes place.

The burners 26 are usually arranged in superposed rows on the longitudinal sides and end faces of the furnace 20, with efforts being made by means of the type and arrangement of the burners 26 to achieve very uniform distribution of inward radiation of heat along the circumference of the reaction tubes 22.

The part of the furnace 20 in which the burners 26 and the reaction tubes 22b are arranged and in which the predominant conversion of the dissociation reaction takes place is referred to as the radiation zone 16. Above the actual reaction tubes 22 and upstream of the radiation zone 16 viewed in the flow direction of the reaction mixture there are further rows of tubes 22s, the tubes 22s of which are preferably arranged horizontally next to one another. These rows of tubes 22s are typically unfinned and largely shield internals 22a located above them, e.g. finned heat exchange tubes 22a of the convection zone 17, against direct radiation from the firing space. In addition, these rows of tubes 22s increase the thermal efficiency of the reaction zone by means of structurally optimized convective heat transfer. In technical language usage, these tubes 22s or rows of tubes 22s are usually referred to as “shock tubes” or “shock zone”.

For the purposes of the invention, the “reaction zone” is made up of the reaction tubes 22b which are located downstream of the shock zone in the flow direction of the reaction gas and are preferably vertically aligned or offset above one another. The major part of the EDC used is converted into VCM here.

The actual dissociation reaction takes place in the gaseous state. Before entering the reaction zone, the EDC is firstly preheated and then vaporized and possibly superheated. Finally, the gaseous EDC enters the reactor where it is usually heated further in the shock tubes 22s and finally enters the reaction zone where the thermal dissociation reaction commences at temperatures above about 400° C.

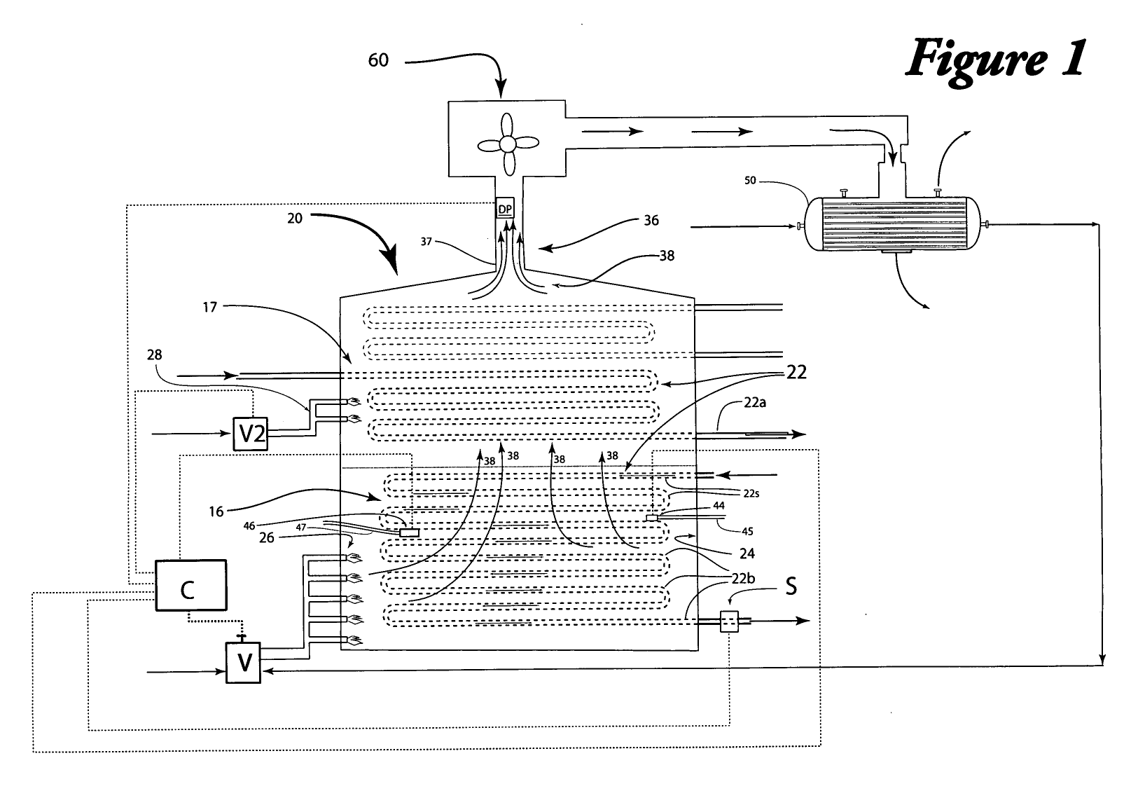

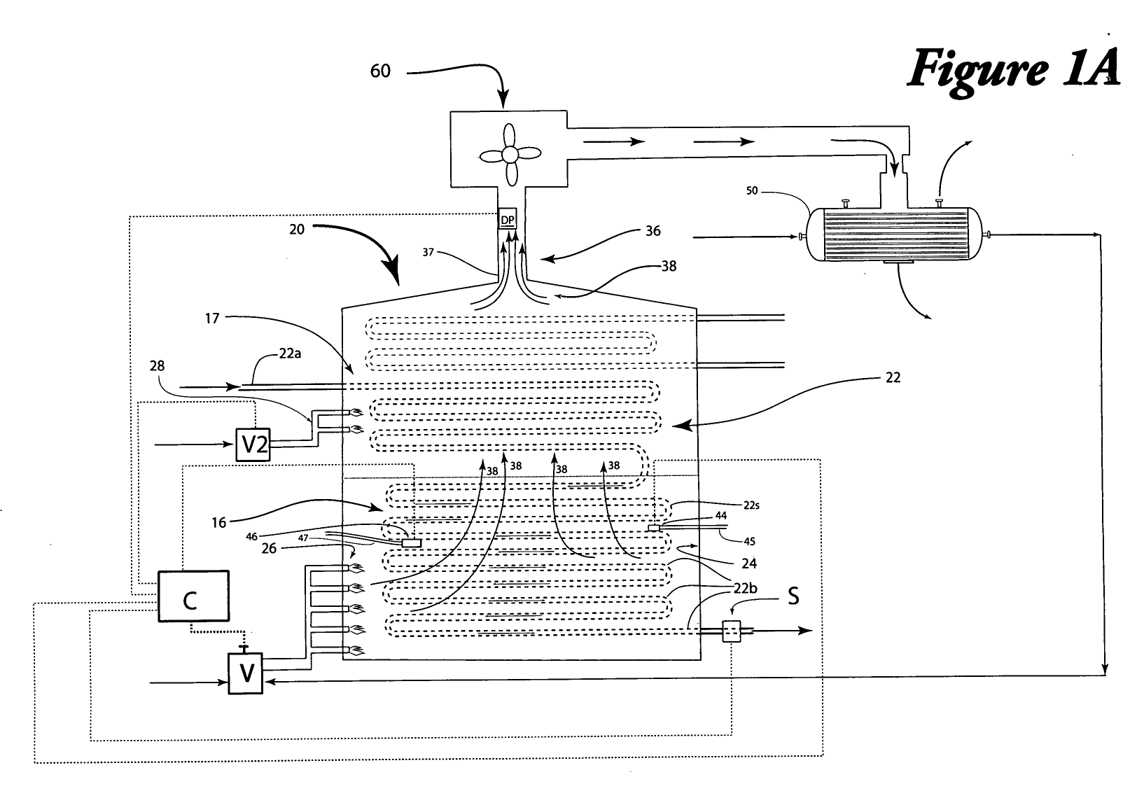

The vaporization of the EDC takes place outside the dissociation furnace 20 in a separate apparatus, viz. the EDC vaporizer 40, in modern plants. The EDC vaporizer 40 is heated by means of steam in some processes. Heating by means of the sensible heat of the reaction mixture leaving the furnace 20 is more economical. In relatively old plants, liquid EDC is introduced into the preheating zone of the furnace 20 and then vaporizes within the furnace 20 as shown in FIG. 1A.

The invention provides a process which comprises vaporization of the feed EDC outside the dissociation furnace 20 by means of a separate apparatus 40.

In the process of the invention, the sensible heat content of the reaction mixture leaving the dissociation furnace 20 is utilized to vaporize the feed EDC before it enters the dissociation furnace 20, i.e. the EDC vaporizer 40 is heated by means of the hot stream leaving the reactor 20, hereinafter referred to as “dissociation gas”, which is cooled in the process but partial or complete condensation of the dissociation gas is avoided. An apparatus as has been described, for example, in EP 276,775 A2 (incorporated herein by reference in its entirety) has been found to be particularly advantageous for this purpose.

Although the major part of the feed EDC is reacted in the reaction zone, EDC is also converted into VCM in the pipe from the outlet of the dissociation furnace 20 to the inlet into the EDC vaporizer 40, with the reaction adiabatically withdrawing heat from the dissociation gas and the dissociation gas being cooled. This proportion of the total conversion, hereinafter referred to as “after-reaction”, proceeds until entry into the EDC vaporizer 40 where the reaction finally ceases when the temperature drops below a certain minimum. The sum of the volumes of the pipe section from the outlet from the dissociation furnace 20 to the inlet of the EDC vaporizer 40 and the dissociation gas side of the EDC vaporizer 40 itself to the outlet port of the EDC vaporizer 40 is, for the purposes of the invention, referred to as “after-reaction zone”.

The heat of the hot flue gas 38 leaving the radiation zone is utilized by convective heat transfer in the convection zone 17 which follows the radiation zone 16 and is physically located above the latter, with, for example, the following operations being able to be carried out:
preheating of liquid EDC
vaporization of preheated EDC
heating of heat transfer media
preheating of boiler feed water
generation of steam
preheating of combustion air
preheating of other media (including media extraneous to the process).

Vaporization of EDC in the tubes 22a located in the convection zone 17 is dispensed with in modern plants since in this mode of operation the vaporizer tubes 22a quickly become blocked by carbon deposits, which adversely affects the economics of the process as a result of shortened cleaning intervals.

The physical combination of radiation zone 16 and convection zone 17 with the associated flue gas chimney 37 is referred to as dissociation furnace 20 by those skilled in the art.

The utilization of the heat content of the flue gas 38, in particular for preheating the EDC, is of central importance for the economics of the process since very complete exploitation of the heat of combustion of the fuel has to be sought.

The reaction mixture leaving the dissociation furnace 20 contains not only the desired product VCM but also HCl (hydrogen chloride) and unreacted EDC. These are separated off in subsequent process steps and recirculated to the process or utilized further. Furthermore, the reaction mixture contains by-products which are likewise separated off, worked up and utilized further or recirculated to the process. These relationships are known to those skilled in the art.

The by-products carbon and tar-like substances which are formed over a plurality of reaction steps from low molecular weight by-products such as acetylene and benzene and deposit in the serpentine tubes 22 of the dissociation furnace 20 (and also in downstream apparatuses such as the EDC vaporizer 40) where they lead to a deterioration in heat transfer and, by constricting the free cross section, to an increase in the pressure drop are of particular importance for the process.

These by-products lead to the plants having to be shutdown and cleaned at regular intervals. Owing to the high costs for the cleaning itself and also the associated loss of production, very long time intervals between cleaning operations are desired.

After exit from the dissociation furnace 20, the sensible heat of the dissociation gas can, as described above, be utilized for vaporizing the feed EDC.

Apparatuses for this purpose are described, for example, in EP 264,065 A1, in DE 103 26 248 A1 or DE 36 30 162 A1. An apparatus corresponding to EP 264,065 A1, in which the feed EDC is vaporized outside the furnace 20 by means of the sensible heat content of the dissociation gas, has been found to be particularly advantageous.

Directly after the utilization of heat by vaporization of feed EDC and cooling of the dissociation gas (in the case of processes in which the heat of the dissociation gas is not recovered, also directly after exit from the dissociation furnace 20), the dissociation gas is scrubbed and cooled further in a quenching column by direct contact with a cool, liquid runback stream or circulated stream. This has the primary purpose of scrubbing out carbon particles present in the dissociation gas or condensing and likewise scrubbing out tar-like substances which are still gaseous since both components would interfere in the subsequent work-up steps.

Finally, the dissociation gas is passed to a work-up by distillation, in which the components hydrogen chloride (HCl), VCM and EDC are separated from one another.

This work-up stage generally comprises at least one column which is operated under superatmospheric pressure and in which pure HCl is obtained as overhead product (hereinafter HCl column).

Recently, there is a tendency toward ever higher production capacities and thus ever increasing plant sizes in the construction of new plants for the preparation of VCM by thermal dissociation of EDC. At the same time, the production volume which can be realized using a dissociation furnace is limited by various factors.

Thus, for example, the pressure drop over the shock tubes 22s and the actual reactor tubes 22b must not be too high, so that the pressure at the top of the HCl column is sufficient to be able to condense the hydrogen chloride with an economically feasible energy usage. The lower limit for this pressure at the top of the column is about 9-11 bar abs.

The space-time yield based on VCM and the reactor volume, i.e. the total volume of the reaction tubes 22, in kg of VCM/(m³ h) depends essentially on the heat flux (dimensions: W/m²), i.e. the quantity of heat which can be transferred per unit area through the tube wall to the reaction mixture flowing through the tube 22, and also on the ratio of the surface area to the volume of the reaction tube 22 (dimensions: m²/m³).

Since the ratio of surface area/volume of the tubes 22 decreases with increasing tube diameter, the space-time yields which can be achieved become ever smaller with increasing diameter of the reactor tubes 22. One possible way of at least partially compensating this effect would be to increase the heat flux. However, this can not be increased beyond a particular limit in conventional processes since otherwise increased by-product formation and greatly accelerated deposition of carbon occur as a result of the high interior wall temperatures of the reaction tube 22. Average heat fluxes of about 28-32 kW/m² are customary in industrial practice.

Due to these restrictions, there is at present an upper limit to the capacity of EDC dissociation furnaces of about 250 000 metric tons per annum of VCM. Larger capacities have to be achieved by connection of two or more furnaces 20 in parallel, i.e. by means of a multistream configuration.

If it were possible to increase the capacity of a dissociation furnace 20 significantly, the capacity limit above which a multistream configuration of the EDC dissociation is necessary could be increased. The saving of one or more furnaces 20 with the associated periphery (including EDC vaporizer, feed preheating, quench column) would give a very substantial economic advantage in the achievement of high plant capacities.

An economic advantage would also be obtained in the case of plant capacities below the limit above which a multistream configuration is necessary. If the space-time yield of the EDC dissociation reaction were to be able to be increased significantly, lower reactor volumes would be possible. In the case of the dissociation furnace 20, this means specifically that fewer reactor tubes 22 or reactor tubes 22 having a smaller diameter would have to be installed. Since these tubes 22 consist of expensive high-temperature materials and make up a significant proportion of the costs for construction of a dissociation furnace 20, it would be possible for a dissociation furnace 20 for a particular plant capacity to be constructed significantly more cheaply than hitherto.

Attempts have for a long time been made to increase the space-time yield of the EDC dissociation by means of various measures. These measures have the aim of increasing the amount of product which can be obtained from a given reactor volume and can be divided into:
use of heterogeneous catalysts
use of chemical promoters
other measures (e.g. injection of electromagnetic radiation).

It is generally assumed that the measures proposed hitherto contribute to physical or chemical initiation to provide free chlorine radicals in the reaction space. The thermal dissociation of EDC is a free-radical chain reaction in which the first step is elimination of a free chlorine radical from an EDC molecule:

C₂H₄Cl₂→C₂H₄Cl+Cl

The high activation energy of this first step compared to the subsequent chain propagation steps is the reason why the dissociation reaction proceeds appreciably only above a temperature of about 420° C.

The use of a heterogeneous catalyst makes elimination of a free chlorine radical from the EDC molecule possible, e.g. by dissociative adsorption of the EDC molecule on the catalyst surface. Very high EDC conversions can be achieved using heterogeneous catalysts. However, decomposition of the VCM and thus carbon formation on the catalyst surface occur on and in the vicinity of the catalyst surface as a result of high local partial pressures of VCM, leading to rapid deactivation of the catalyst. Owing to the frequent regenerations made necessary thereby, heterogeneous catalysts have hitherto not been employed in the large-scale production of VCM.

In the case of physical measures, e.g. irradiation with short-wavelength light, the energy for elimination of the free chlorine radical is provided from an external source. Thus, adsorption of a quantum of short-wavelength light by the EDC molecule provides the energy for elimination of the free chlorine radical:

C₂H₄Cl₂+hν→C₂H₄Cl+Cl

where “ν” it indicates the frequency of a photon. When chemical initiators are used, a chlorine atom is eliminated from the EDC molecule by reaction of the EDC with the initiator or the free chlorine radicals are provided by decomposition of the initiator. Chemical initiators are, for example, elemental chlorine, bromine, iodine, elemental oxygen, chlorine compounds such as carbon tetrachloride (CCl₄) or chlorine-oxygen compounds such as hexachloroacetone.

All the measures for initiating the reaction bring about a significant reduction in the temperature level in the reactor at a given conversion or a large increase in the conversion at a given temperature level.

Comprehensive literature is available on the use of catalysts for thermal dissociation of EDC. An example which may be mentioned is EP 002,021 A1.

The high tendency of catalysts to become carbonized and the need for frequent regeneration stand in the way of the use of these in industrial practice.

Physical measures such as injection of electromagnetic radiation into the reaction tube 22 (described, for example, in DE 30 08 848 A1 or DE 29 38 353 A1) have also not found their way into industrial practice despite their suitability in principle. The reasons for this may well be related to safety (since, for example, a pressure-resistant optical window is necessary for input of light). Further physical measures which have been described, for instance injection of a heated gas into the reaction mixture (WO 02/094,743 A2) have also not been used hitherto on an industrial scale.

DE 103 19 811 A1 describes the electromagnetic and photolytic induction of free-radical reactions. In addition, this document describes an apparatus for introducing this energy into a reactor. Although this document mentions the use of dissociation promoters in general terms, no information can be found there about the design and the operation of the reactor used.

The use of chemical promoters is in principle the least technically complicated because it is neither necessary to fill the reactor with catalyst (facilities for filling/emptying and regeneration are required) nor are additional facilities for injection of electromagnetic radiation required. The promoter can be introduced into the feed EDC stream in a simple manner.

Increasing the conversion of the EDC dissociation by addition of halogens or halogen-releasing compounds has been described by Barton et al. (U.S. Pat. No. 2,378,859 A), where the experiments of fundamental importance were carried out at atmospheric pressure in a glass apparatus. Krekeler (DE patent No. 857,957) has described a process for the thermal dissociation of EDC under superatmospheric pressure. Carrying out the reaction at superatmospheric pressure is of fundamental importance for large-scale industrial use since only then is economical fractionation of the reaction mixture possible. This relationship is known to those skilled in the art. Krekeler also recognized the problem of accelerated formation of carbon deposits at high conversions and nominated 66% as a practical upper limit to the conversion. In DE-B-1,210,800, Schmidt et al. describe a process in which operation at superatmospheric pressure is combined with the addition of a halogen. Here, conversions of about 90% are achieved at working temperatures of 500-620° C. Schmidt et al. also stated that the conversion reaches saturation as a function of the amount of halogen added, i.e. that a significant increase in conversion is no longer achieved above a particular amount of halogen added relative to the feed EDC stream.

The simultaneous addition of halogen or other chemical promoters at least two points on the reactor tube 22 has been described by Sonin et al. in DE 1 953 240 A. Here, conversions in the range from 65 to 80% were achieved at reaction temperatures of 250-450° C.

In DE 2 130 297 A, Scharein et al. describe a process for the thermal dissociation of EDC under superatmospheric pressure, in which chlorine is introduced at a plurality of points on the reactor tube 22. Here, conversion of 75.6% (example 1) or 70.5% (example 2) are achieved at a reaction temperature of 350-425° C. This publication also refers to the importance of the ratio of surface area/volume of the reactor 20 and to the importance of the loading of the heating areas (heat flux).

The problem of rapid carbonization of the reactor at high conversions of the dissociation reaction is avoided in a process disclosed by Demaiziere et al. in U.S. Pat. No. 5,705,720 A by diluting the gaseous EDC entering the reactor with hydrogen chloride. Here, hydrogen chloride is added to the EDC in a molar ratio of from 0.1 to 1.8. At the same time, dissociation promoters can also be added to the mixture of EDC and HCl according to this process. Since the VCM partial pressure is kept low by the dilution with large amounts of HCl, high conversions can be achieved without carbonization of the reactor. However, disadvantages here are the energy input for heating and the subsequent removal of the HCl added for dilution.

In U.S. Pat. No. 4,590,318 A, Longhini discloses a process in which a promoter is introduced into the dissociation gas after exit from the dissociation furnace 20, i.e. into the after-reaction zone 42. Here, the heat content of the dissociation gas is exploited in order to increase the total conversion of the EDC dissociation. However, this method is inferior to measures for increasing the space-time yield in the dissociation furnace 20 itself since only the heat still present in the dissociation gas stream after exit from the dissociation furnace 20 can be exploited and the usable quantity of heat is limited when the heat of the dissociation gas stream is to be utilized for vaporization of the feed EDC.

Felix et al. (EP 0 133 699 A1), Wiedrich et al. (U.S. Pat. No. 4,584,420 A) and Mielke (DE 42 28 593 A1) teach the use of chlorinated organic compounds instead of chlorine as dissociation promoters. This makes it possible in principle to achieve the same effects on the EDC dissociation reaction as when using elemental halogens such as chlorine or bromine. However, since these are materials which are frequently not (like chlorine, available in the integrated facility for VCM production, they have to be introduced separately into the process, which in turn is associated with increased costs for procuring them and disposal of the residues.

DE 102 19 723 A1 relates to a process for metered addition of dissociation promoters in the course of preparation of unsaturated halogenated hydrocarbons. This document does not disclose any further details regarding the thermal design of the reactor.

Although the effects of dissociation promoters on the reaction of thermal dissociation of EDC and their main advantages have been known for a relatively long time, the use of dissociation promoters has hitherto not found its way into the commercial production of VCM by thermal dissociation.

This is because all previously disclosed processes aim at increased conversions of the dissociation reaction (at least 65%), although it was recognized early on (DE patent 857 957) that a significantly increased tendency for carbon deposits to be formed in the reactor tubes 22 and the after-reaction zone 42 has to be expected above this limit. The increased tendency for formation of carbon deposits, which has hitherto prevented the use of dissociation promoters in industrial practice is due not to the promoters themselves but to a combination of higher VCM partial pressures in the reaction mixture (as occur at conversions above 65%) with high temperatures of the dissociation gas and the interior wall of the reactor tube 22. This hypothesis is also supported, in particular, by the results disclosed in U.S. Pat. No. 5,705,720 A where high conversions can be achieved with and without dissociation promoter by dilution of the reaction mixture with relatively large amounts of HCl, without an increased tendency for carbon deposits to be formed occurring.

In conventional processes, the use of promoters has always been associated with an increase in conversion and has hitherto led to dissociation promoters not having found their way into the large-scale preparation of VCM by thermal dissociation of EDC.

The problem is thus to exploit the properties of dissociation promoters in such a way that the space-time yield in the reaction zone of the dissociation furnace 20 is significantly increased, with the intervals between necessary cleaning not being shorter than in the case of a plant of the same production capacity without use of promoters and with the heat content of the dissociation gas being utilized to vaporize the feed.

It is an object of the present invention to provide a reactor having a capacity which is significantly increased over that of conventional plants. The above-described advantages can be achieved in this way.

A further object of the present invention is to provide a process for the thermal dissociation of halogenated aliphatic hydrocarbons, in which significantly increased space-time yields compared to conventional processes can be achieved and which has a reduced tendency for carbon deposits to be formed.

The invention provides a process for the thermal dissociation of halogenated aliphatic hydrocarbons to form ethylenically unsaturated halogenated hydrocarbons in a reactor which comprises reaction tubes 22 running through a convection zone 17 and through a radiation zone 16 located downstream in the flow direction of the reaction gas with upstream shock tubes 22S, with burners being provided in the radiation zone in order to introduce thermal energy into the shock tubes 22s and reaction tubes 22b, and comprises a heating apparatus 40 for the halogenated aliphatic hydrocarbon (“feed”) which is located outside the reactor 20 and is heated by the energy content of the reaction gases leaving the radiation zone 16, wherein
a) a chemical promoter for the thermal dissociation is introduced into the reaction tubes 22b and/or localized energy input to promote the thermal dissociation into the reaction tubes 22b is effected at one or more points within the reactor 20,
b) the amount of the chemical promoter and/or the intensity of the localized energy input to form free radicals in the reaction tubes 22b is selected so that at least 50%, preferably at least 70%, of the halogenated aliphatic hydrocarbon used can be vaporized by the energy content of the reaction gases leaving the radiation zone 16 without condensation of the reaction gases leaving the radiation zone 16 occurring,
c) the heat exchange area in the radiation zone 16, defined as the sum of the surface area of the shock tubes 22s and the surface area of the reaction tubes 22b, is dimensioned so that the average heat flux through the heat exchange area of the radiation zone 16 is at least 35 kW/m², preferably at least 40 kW/m², and
d) the conversion of the dissociation reaction, based on the halogenated aliphatic hydrocarbon used, is in the range from 50 to 65%.

The invention further provides an apparatus for the thermal dissociation of halogenated aliphatic hydrocarbons to form ethylenically unsaturated halogenated hydrocarbons, which comprises a reactor 20 which comprises reaction tubes 22 running through a convection zone 17 and through a radiation zone 16 located downstream in the flow direction of the reaction gas with upstream shock tubes 22s, with burners 26 being provided in the radiation zone 16 in order to introduce thermal energy into the shock tubes 22s and reaction tubes 22b, and comprises a heating apparatus 40 for the halogenated aliphatic hydrocarbon (“feed”) which is located outside the reactor 20 and is heated by the energy content of the reaction gases leaving the radiation zone 16, comprising the elements:
A) means 44 of introducing chemical promoters for the thermal dissociation into the reaction tubes 22 and/or means 46 of introducing localized energy to promote the thermal dissociation at one or more points on the reaction tubes 22,
B) means of selecting the amount of the chemical promoter and/or the intensity of the localized energy input to form free radicals in the reaction tubes 22 in such a way that at least 50%, preferably at least 70%, of the halogenated aliphatic hydrocarbon used can be vaporized by the energy content of the reaction gases leaving the radiation zone 16 without condensation of the reaction gases leaving the radiation zone 16 occurring,
C) heat exchange areas in the radiation zone 16, defined as the sum of the surface area of the shock tubes 22s and the surface area of the reaction tubes 22b, which are dimensioned so that the average heat flux through the heat exchange area of the radiation zone 16 is at least 35 kW/m².

It has surprisingly been found that the production quantity which can be achieved using dissociation reactors 20 of a given size can be increased considerably when the heat exchange areas are dimensioned so that heat fluxes above 35 kW/m² are obtained and initiating measures are used to reduce the reaction temperature and the interior wall temperature of the reaction tube 22b. At the same time, the feed stream and the heating power of the reaction furnace 20 are increased so that the conversion of the reaction is not significantly increased compared to processes without use of initiating measures. To be able to continue to operate the process economically despite the reduction in the reaction temperature, the process parameters have to be set so that at least 50% of the amount of feed used is vaporized by means of the sensible heat content of the reaction mixture leaving the reaction zone 16.

In a preferred embodiment of the process of the invention, the flue gas 38 is condensed in a heat exchanger and the waste heat from the flue gas 38 is utilized for preheating the burner air or other media, e.g. liquid starting material, as additional measure.

In the process variant, the heat from the cooling of the flue gas 38 to below its dew point and the heat of condensation of the water vapor present in the flue gas 38 are utilized.

In the case of this measure, heat exchange preferably occurs at the point at which the flue gas 38 leaves the convection zone 17.

This measure is employed especially in the case of fuels having a low proportion of acid-forming components. However, it can also be used in the case of fuels having a moderate to high proportion of acid-forming components.

In this process variant, the apparatus of the invention comprises D) at least one heat exchanger 50 which is used for recovering waste heat from the condensation of the flue gas 38 for preheating the combustion air or other media, e.g. liquid starting material.

The consumption of fuel of a dissociation furnace 20 at a given efficiency of the dissociating process can likewise be reduced considerably by the measure of recovering the latent waste heat present in the flue gas and preheating the combustion air.

The introduction of chemical promoters for the thermal dissociation can be effected at any points. The promoter can be added to the feed, preferably the gaseous feed. The promoter is preferably introduced into the shock tubes 22s or in particular reaction tubes 22b in the radiation zone 16.

The localized energy input to promote the thermal dissociation is effected into the reaction tubes 22b at one or more points within the reactor 20.

The process of the invention is described by way of example for the EDC/VC system. It is also suitable for preparing other halogen-containing unsaturated hydrocarbons from halogen-containing saturated hydrocarbons. In all these reactions, the dissociation is a free-radical chain reaction in which not only the desired product but also undesirable by-products which on long-term operation lead to carbon deposits in the plants are formed. Preference is given to the preparation of vinyl chloride from 1,2-dichloroethane.

For the purposes of the present description, “localized energy input into the reaction tubes to promote the thermal dissociation” refers to physical measures which are able to initiate the dissociation reaction. Such measures can be, for example, injection of high-energy electromagnetic radiation or local introduction of thermal or nonthermal plasmas, e.g. hot inert gases.

For the purposes of the present description, the “average heat flux through the heat exchange area of the radiation zone” is the total quantity of heat transferred through the heat exchange area of the radiation zone 16 divided by the heat exchange area of the radiation zone 16. According to the invention, this is at least 35 kW/m².

For the purposes of the present description, the expression “without condensation of the reaction gases leaving the radiation zone occurring” means that neither partial condensation or total condensation of the reaction gas occurs.

Means 44 of introducing chemical promoters for the thermal dissociation are known to those skilled in the art. These are generally feed lines which allow introduction of predetermined amounts of chemical promoters into the feed gas stream or feed lines 45 which allow the introduction of predetermined amounts of chemical promoters into the reaction tubes 22b at the level of the radiation zone 16. These feed lines 45 can have nozzles at the reactor end. Preference is given to one or more of these feed lines 45 opening into the tubes 22b in the first third, viewed in the flow direction of the reaction gas, of the radiation zone 16.

Means 46 of introducing localized energy into the reaction tubes 22b at one or more points in the radiation zone 16 to promote the thermal dissociation are likewise known to those skilled in the art. These can likewise be feed lines 47 which may have nozzles at the reactor end and via which thermal or nonthermal plasma is introduced into the reaction tubes 22b at the level of the radiation zone 16; or they can be windows via which electromagnetic radiation or particle beams are injected into the reaction tubes 22b at the level of the radiation zone 16. Preference is given to one or more of these feed lines 47 opening into the tubes 22b in the first third, viewed in the flow direction of the reaction gas, of the radiation zone 16; or the windows for injection of the radiation being installed in the first third.

Ways of selecting the amount of the chemical promoter and/or the intensity of the localized energy input into the reaction tubes 22b to form free radicals are likewise known to those skilled in the art. These are generally regulating circuits in which a command variable is used to regulate the amount or intensity. As command variables, it is possible to use all process parameters by means of which it is possible to draw conclusions as to the energy content of the reaction gases leaving the radiation zone 16. Examples are the temperature of the exiting reaction gases, the content of dissociation products in the reaction gases or the wall temperature of the reaction tubes 22b at selected places.

The dimensions of the heat exchange areas in the radiation zone 16 can be determined by a person skilled in the art by means of routine tests.

The above-described combination of measures or features make greatly increased space-time yields, compared to conventional processes or apparatuses, possible without the disadvantages known from the literature, e.g. increased formation of by-products and a strong tendency for carbon deposits to be formed, occurring.

At one or more points on the shock tubes 22s or the tubes 22b in the reaction zone, electromagnetic radiation of a suitable wavelength or a particle beam is radiated in or a chemical promoter is added or a combination of these measures is undertaken. In the case of the addition of a chemical promoter, the addition can also be into the feed line for the gaseous feed, for example into the EDC from the EDC vaporizer 40, before entry into the dissociation furnace 20.

The localized energy input to form free radicals is preferably effected by electromagnetic radiation or particle beams; particular preference is given here to ultraviolet laser light.

In the case of addition of a chemical promoter, the use of elemental halogen, in particular elemental chlorine, is preferred.

The chemical promoter can be diluted with a gas which is inert toward the dissociation reaction, with the use of hydrogen chloride being preferred. The amount of inert gas used as diluent should not exceed 5 mol % of the feed stream.

The intensity of the electromagnetic radiation or the particle beam or the amount of the chemical promoter is set so that the molar conversion, based on the feed, at the dissociation gas-end outlet of the feed vaporizer 40 is in the range from 50 to 65%, preferably from 52 to 57%.

Particular preference is given to a molar conversion, based on the EDC used, at the dissociation gas-end outlet of the feed vaporizer 40 of 55%.

The temperature of the reaction mixture leaving the reactor 20 is preferably in the range from 400° C. to 470° C.

The heat exchange area, defined as the sum of the external surface areas of the (unfinned) shock tubes 22s and the tubes 22b in the reaction zone, is dimensioned so that the average heat flux, defined as the quotient of the total heat transferred to the dissociation gas in the radiation zone 16 and the sum of the external surface area of the unribbed shock tubes 22s and the tubes 22b in the reaction zone, is at least 35 kW/m².

Preference is given to the heat exchange area being dimensioned so that the average heat flux, defined as the quotient of the total heat transferred to the dissociation gas in the radiation zone 16 and the sum of the external surface area of the unfinned shock tubes 22s and the tubes 22b in the reaction zone, is in the range from 40 kW/m² to 80 kW/m², particularly preferably from 45 kW/m² to 65 kW/m².

The process of the invention is particularly preferably used for the thermal dissociation of 1,2-dichloroethane to form vinyl chloride.

High space-time yields are achieved by means of the process of the invention. These are preferably, based on the volume of the reaction tube 22, defined as sums of the volumes of the shock tubes 22s and the reaction tubes 22b, from the inlet into the radiation zone 16 of the reactor 20 to the outlet from the radiation zone 16 of the reactor 20, at least 2000 kg, preferably from 3000 to 6000 kg, of ethylenically unsaturated halogenated hydrocarbons, preferably vinyl chloride, per hour and cubic meter (kg/m³·hr).

The process of the invention includes not only the thermal dissociation of halogenated, aliphatic hydrocarbons in the actual dissociation furnace 20 but also, as further process step, the vaporization of the liquid feed, for example the liquid EDC, before entry into the radiation zone 16 of the dissociation furnace 20. These measures have to be taken into account together with the actual thermal dissociation or the operation of the dissociation furnace 20 in order to determine the economics of the dissociation process.

A preferred embodiment of the invention is directed to a process in which the sensible heat of the dissociation gas is exploited in order to vaporize liquid, preheated feed, e.g. EDC, before entry into the radiation zone 16, preferably using a heat exchanger 40 as has already been described in EP 276,775 A2. Particular attention should here be given to ensuring that firstly the dissociation gas is still hot enough on leaving the dissociation furnace 20 to vaporize the total amount of the feed by means of its sensible heat content and secondly the temperature of the dissociation gas on entering this heat exchanger 40 does not go below a minimum value in order to prevent condensation of tar-like substances in the heat exchanger tubes.

In a further preferred embodiment of the vaporization of the feed, which has likewise been described in EP 276,775 A2, the temperature of the dissociation gas at the exit from the dissociation furnace 20 is so low that the heat content of the dissociation gas is not sufficient to vaporize the feed completely. In this embodiment of the invention, the missing proportion of gaseous feed is produced by flash evaporation of liquid feed in a vessel, preferably in the steaming-out vessel of a heat exchanger 40, as has been described in EP 276,775 A2. In this case, preheating of the liquid feed advantageously occurs in the convection zone 17 of the dissociation furnace 20. In this embodiment of the invention, too, it has to be ensured that the temperature of the dissociation gas at the inlet into this heat exchanger 40 does not go below a minimum value in order to prevent condensation of tar-like substances in the heat exchanger tubes.

The heat content of the dissociation gas is used to vaporize at least 50% of the feed by means of indirect heat exchange without the dissociation gas condensing either partly or completely.

As heat exchanger 40, preference is given to using an apparatus as is described, for example, in EP 264,065 A1. Here, liquid halogenated aliphatic hydrocarbon is heated indirectly by the hot product gas comprising the ethylenically unsaturated halogenated hydrocarbon which leaves the reactor 20, vaporized and the resulting gaseous feed gas is introduced into the reactor 20, with the liquid halogenated aliphatic hydrocarbon being heated to boiling by the product gas in a first vessel 52 and from there being transferred to a second vessel 54 in which it is partly vaporized without further heating under a pressure which is lower than in the first vessel 52 and the vaporized feed gas being fed into the reactor 20 and the unvaporized halogenated aliphatic hydrocarbon being recirculated to the first vessel 52.

In a particularly preferred variant of this process, the halogenated aliphatic hydrocarbon is heated in the convection zone 17 of the reactor 20 by means of the flue gas 38 produced by the burners 26 which heat the reactor 20 before being fed into the second vessel.

Particular preference is given to a mode of operation in which the entire feed is vaporized by indirect heat exchange with the dissociation gas without the dissociation gas being either partly or completely condensed.

If the feed is not vaporized completely by means of the heat content of the dissociation gas, the residual amount of feed is preferably vaporized by flash evaporation into a vessel, with the feed being preheated beforehand in the liquid state in the convection zone of the dissociation furnace. As vessel for the flash evaporation, preference is given to using the steaming-out vessel of a heat exchanger, as has been described, for example, in EP 264,065 A1.

In a further preferred variant of the process of the invention, the temperature of the reaction gas entering the heating apparatus 40 as shown in FIG. 1 of EP 264,065 A1 located outside the reactor 20 is measured and serves as command variable for regulation of the amount of chemical promoter added and/or the intensity of the localized energy input. Of course, other measured parameters can also be employed as command variable, for example the content of products of the dissociation reaction.

In a further preferred process variant, the molar conversion of the dissociation reaction is determined downstream of the point at which the dissociation gas leaves the EDC vaporizer or at the top of the quenching column, for example by means of an on-line analytical apparatus, preferably by means of an on-line gas chromatograph.

In a further preferred variant of the process of the invention, the flue gas 38 is extracted by means of a flue gas blower 60 after leaving the convection zone 17 and is passed through one or more heat exchangers 50 where it is condensed. The waste heat is utilized for heating the burner air. The condensate formed is, if appropriate, worked up and discharged from the process. The remaining gaseous constituents of the flue gas are, if appropriate, purified and released into the atmosphere.

Particular preference is given to a process in which the flue gas 38 to be cooled to below the dew point is introduced in a downward direction from above into the heat exchanger 50 provided for this purpose, after cooling leaves the heat exchanger 50 in the upward direction and the condensate formed can freely runoff downward from the heat exchanger 50 and is thus completely separated off from the flue gas stream.

The amount of fuel can be divided in either equal parts or unequal parts over the burner rows 26 in the furnace.

It is possible to use reactor tubes 22 having an internal diameter of at least 200 mm, preferably from 250 to 350 mm. However, the internal diameter of the reactor tubes 22 is not restricted to these dimensions.

When dissociation promoters are used and/or physical measures are employed for initiating the dissociation reaction of the feed, the process of the invention makes it possible to employ high average heat fluxes and avoids the disadvantages which usually occur at high space-time yields in the thermal dissociation of the feed.

The advantage of the process lies, in particular, in the fact that when setting moderate conversions, which correspond to those of “conventional” processes, using promoters, it is possible to set comparatively very high heat fluxes and thus transfer large heat flows to the dissociation gas without the formation rates of by-products or carbon deposits being increased. The reason for this is that the addition of promoters and/or the use of physical measures to initiate the dissociation reaction significantly reduce the overall temperature level in the reaction space and also the interior wall temperature of the reactor tube 22, as a result of which the reaction mixture is subjected to mild conditions despite high transferred heat flows.

Since the smaller reactor volumes compared to conventional processes (equivalent to shorter tube lengths in the radiation zone) result in lower flow pressure drops, reactors designed according to the invention can be supplied with comparatively large amounts of feed without the pressure dropping below the minimum pressure at the inlet into the HCl column which is necessary for economical fractionation of the reaction mixture.

A further advantage is that it is also possible to achieve reactor tube 22 diameters which are not possible in conventional processes since excessively high internal wall temperatures would otherwise occur as a result of their low surface area/volume ratio.

The economics of the process are also influenced by the sum of the pressure drops over the dissociation furnace 20 (comprising convection zone 17 and radiation zone 16), the heat exchanger 40 for vaporization of the feed and also any quenching system (“quenching column”) present. This should be as low as possible since when the dissociation products are separated off by distillation, they have to be condensed at the top of a column using a refrigeration machine for cooling the condenser. The greater the sum of the pressure drops over the total system for “thermal dissociation”, the lower the pressure of the top of the column and the dissociation product separated off, for example HCl, has to be condensed at a correspondingly lower temperature. This leads to an increased specific energy consumption by the refrigeration machine, which in turn has an adverse effect on the economics of the total process.

The invention is illustrated below with the aid of examples. No limitation is intended thereby.

### Example 1

42 500 kg/h of gaseous EDC were passed through a serpentine tube 22 having a length of 232 m and an internal diameter of 153.4 mm at a pressure of 21 bar abs. and an inlet temperature of 360° C. in a dissociation furnace 20. At the reactor inlet, a mixture of 42.5 kg/h of chlorine (corresponding to 1000 ppm by weight) and 250 kg/h of hydrogen chloride was introduced into the gaseous EDC. The reactor volume was 4.3 m³. The fired power was 10 000 kW. The temperature of the dissociation gas at the outlet from the furnace 20 was 418° C.; the conversion was 52.5%. The temperature of the flue gas 38 at the outlet from the radiation zone 16 was 897° C. The thermal power absorbed was 5113 kW, and the average heat flux was 42 kW/m². The reactor output was 3270 kg of VCM/m³ h.

### Example 2

64 000 kg/h of gaseous EDC were passed through a serpentine tube 22 having a length of 232 m and an internal diameter of 153.4 mm at a pressure of 21 bar abs. and an inlet temperature of 360° C. in a dissociation furnace 20. At the reactor inlet, a mixture of 64 kg/h of chlorine (corresponding to 1000 ppm by weight) and 250 kg/h of hydrogen chloride was introduced into the gaseous EDC. The reactor volume was 4.3 m³. The fired power was 20 000 kW. The temperature of the dissociation gas at the outlet from the furnace 20 was 440° C.; the conversion was 52.8%. The temperature of the flue gas 38 at the outlet from the radiation zone 16 was 1074° C. The thermal power absorbed was 8220 kW, and the average heat flux was 67 kW/m². The reactor output was 4960 kg of VCM/m³ h.

### Example 3

36 160 kg/h of gaseous EDC were passed through a serpentine tube 22 having a length of 130 m and an internal diameter of 153.4 mm at a pressure of 21 bar abs. and an inlet temperature of 360° C. in a dissociation furnace 20. At the reactor inlet, a mixture of 36.1 kg/h of chlorine (corresponding to 1000 ppm by weight) and 250 kg/h of hydrogen chloride was introduced into the gaseous EDC. The reactor volume was 2.4 m³. The fired power was 10 000 kW. The temperature of the dissociation gas at the outlet from the furnace 20 was 433° C.; the conversion was 52.7%. The temperature of the flue gas 38 at the outlet from the radiation zone 16 was 997° C. The thermal power absorbed was 4550 kW, and the average heat flux was 72 kW/m². The reactor output was 5010 kg of VCM/m³ h.

### Example 4

### Comparative Example, Conventional Process

36 160 kg/h of gaseous EDC were passed through a serpentine tube 22 having a length of 403 m and an internal diameter of 153.4 mm at a pressure of 21 bar abs. and an inlet temperature of 360° C. in a dissociation furnace 20. The reactor volume was 7.5 m³. The fired power was 10 000 kW. The temperature of the dissociation gas at the outlet from the furnace 20 was 490° C.; the conversion was 52.8%. The temperature of the flue gas 38 at the outlet from the radiation zone 16 was 866° C. The thermal power absorbed was 5290 kW, and the average heat flux was 25 kW/m². The reactor output was 1606 kg of VCM/m³ h.

1-27. (canceled)

28. An improved process for the thermal dissociation of a halogenated aliphatic hydrocarbon to form ethylenically unsaturated halogenated hydrocarbons in a reactor which comprises reaction tubes running through a convection zone and through a radiation zone located downstream in the flow direction of the reaction gas with upstream shock tubes, with burners being provided in the radiation zone in order to introduce thermal energy into the shock and reaction tubes, and comprises a heating apparatus for the halogenated aliphatic hydrocarbon which is located outside the reactor and is heated by the energy content of the reaction gases leaving the radiation zone, wherein the improvement comprises:
configuring the reactor and energy input thereto such that the heat exchange area in the radiation zone, defined as the sum of the surface area of the shock tubes and the surface area of the reaction tubes, is dimensioned such that the average heat flux through the heat exchange area of the radiation zone is at least 35 kW/m²;
introducing a controlled input of an initiator for the thermal dissociation of said halogenated aliphatic hydrocarbon into the reaction tubes at one or more points within the reactor, said initiator being chosen from the group consisting of at least one chemical promoter for the thermal dissociation reaction; a localized energy input adapted to form free radicals to promote the thermal dissociation reaction; and combinations of the foregoing:
controlling the input of initiator so that:
at least 50% of the halogenated aliphatic hydrocarbon used can be vaporized by the energy content of the reaction gases leaving the radiation zone without condensation of the reaction gases leaving the radiation zone, and
the conversion of the dissociation reaction, based on the halogenated aliphatic hydrocarbon used, is in the range from 50 to 65%.

29. The process as claimed in claim 28, wherein a localized energy input to form free radicals is effected by means of electromagnetic radiation or by means of a particle beam.

30. The process as claimed in claim 29, wherein the electromagnetic radiation is ultraviolet laser light.

31. The process as claimed in claim 28, wherein elemental chlorine is used as chemical promoter.

32. The process as claimed in claim 31, wherein the elemental chlorine is diluted with hydrogen chloride, with the amount of the hydrogen chloride used for dilution being not more than 5 mol % of the halogenated aliphatic hydrocarbon stream used.

33. The process as claimed in claim 32, wherein the temperature of the reaction mixture leaving the reactor is in the range from 400° C. to 470° C.

34. The process as claimed in 33, wherein the average heat flux in the radiation zone is in the range from 45 to 65 kW/m².

35. The process as claimed in claim 34, wherein the conversion based on the halogenated aliphatic hydrocarbon used is in the range from 52% to 57%.

36. The process as claimed in claim 35, wherein the halogenated aliphatic hydrocarbon is 1,2-dichloroethane and the ethylenically unsaturated halogenated hydrocarbon is vinyl chloride.

37. The process as claimed in claim 36, wherein the space-time yield based on the volume of the reaction tube from the inlet into the radiation zone of the reactor to the outlet from the radiation zone of the reactor is at least 2000 kg of vinyl chloride per hour and cubic meter (kg/m³·hr).

38. The process as claimed in claim 37, wherein liquid halogenated aliphatic hydrocarbon is heated indirectly by the hot product gas comprising the ethylenically unsaturated halogenated hydrocarbon which leaves the reactor, vaporized and the resulting gaseous feed gas is introduced into the reactor, with the liquid halogenated aliphatic hydrocarbon being heated to boiling by the product gas in a first vessel and from there being transferred to a second vessel in which it is partly vaporized without further heating under a pressure which is lower than in the first vessel and the vaporized feed gas being fed into the reactor and the unvaporized halogenated aliphatic hydrocarbon being recirculated to the first vessel.

39. The process as claimed in claim 38, wherein the halogenated aliphatic hydrocarbon is heated by the flue gas produced by the burners which heat the reactor in the convection zone of the reactor before being fed into the second vessel.

40. The process as claimed in claim 39, wherein the temperature of the reaction gas entering the heating apparatus located outside the reactor is measured and serves as command variable for regulating the amount of the chemical promoter added and/or for the intensity of the localized energy input.

41. The process as claimed in claim 40, wherein the conversion of the dissociation reaction is determined downstream after exit of the dissociation gas from the heating apparatus for the halogenated aliphatic hydrocarbon or at the top of the quenching column, preferably by means of an on-line analytical method, in particular by means of an on-line gas chromatograph.

42. The process as claimed in claim 41, wherein the flue gas is condensed in a heat exchanger and the waste heat of the flue gas utilized for preheating the burner air or other media.

43. The process as claimed in claim 42, wherein the flue gas to be cooled to below the dew point is introduced in a downward direction from above into the heat exchanger provided for this purpose, after cooling leaves the heat exchanger in the upward direction and the condensate formed can freely runoff downward from the heat exchanger and is thus completely separated off from the flue gas stream.

44. The process as claimed in claim 42, wherein the heat exchange is effected at the point at which the flue gas leaves the convection zone.

45. The process as claimed in claim 44, wherein the flue gas is extracted by means of a flue gas blower after leaving the convection zone and is passed through one or more heat exchangers where it is condensed, the waste heat is utilized for heating the burner air, the condensate formed is, if appropriate, worked up and discharged from the process, and the remaining gaseous constituents of the flue gas are, if appropriate, purified and released into the atmosphere.

46. The process as claimed in 45, wherein the flue gas to be cooled to below the dew point is introduced in a downward direction from above into the heat exchanger provided for this purpose, after cooling leaves the heat exchanger in the upward direction and the condensate formed can freely runoff downward from the heat exchanger and is thus completely separated off from the flue gas stream.

47. The process as claimed in claim 28, wherein the temperature of the reaction mixture leaving the reactor is in the range from 400° C. to 470° C.

48. The process as claimed in claim 47, wherein elemental chlorine is used as chemical promoter.

49. The process as claimed in claim 48, wherein the elemental chlorine is diluted with hydrogen chloride, with the amount of the hydrogen chloride used for dilution being not more than 5 mol % of the halogenated aliphatic hydrocarbon stream used.

50. The process as claimed in 28, wherein the average heat flux in the radiation zone is in the range from 45 to 65 kW/m².

51. The process as claimed in claim 50, wherein elemental chlorine is used as chemical promoter.

52. The process as claimed in claim 51, wherein the elemental chlorine is diluted with hydrogen chloride, with the amount of the hydrogen chloride used for dilution being not more than 5 mol % of the halogenated aliphatic hydrocarbon stream used.

53. The process as claimed in claim 28, wherein the conversion based on the halogenated aliphatic hydrocarbon used is in the range from 52% to 57%.

54. The process as claimed in claim 47, wherein elemental chlorine is used as chemical promoter.

55. The process as claimed in claim 54, wherein the elemental chlorine is diluted with hydrogen chloride, with the amount of the hydrogen chloride used for dilution being not more than 5 mol % of the halogenated aliphatic hydrocarbon stream used

56. The process as claimed in claim 28, wherein the space-time yield based on the volume of the reaction tube from the inlet into the radiation zone of the reactor to the outlet from the radiation zone of the reactor is at least 2000 kg of vinyl chloride per hour and cubic meter (kg/m³·hr).

57. The process as claimed in claim 28, wherein liquid halogenated aliphatic hydrocarbon is heated indirectly by the hot product gas comprising the ethylenically unsaturated halogenated hydrocarbon which leaves the reactor, vaporized and the resulting gaseous feed gas is introduced into the reactor, with the liquid halogenated aliphatic hydrocarbon being heated to boiling by the product gas in a first vessel and from there being transferred to a second vessel in which it is partly vaporized without further heating under a pressure which is lower than in the first vessel and the vaporized feed gas being fed into the reactor and the unvaporized halogenated aliphatic hydrocarbon being recirculated to the first vessel.

58. The process as claimed in claim 57, wherein the halogenated aliphatic hydrocarbon is heated by the flue gas produced by the burners which heat the reactor in the convection zone of the reactor before being fed into the second vessel.

59. The process as claimed in claim 28, wherein the temperature of the reaction gas entering the heating apparatus located outside the reactor is measured and serves as command variable for regulating the amount of the chemical promoter added and/or for the intensity of the localized energy input.

60. The process as claimed in claim 28, wherein the conversion of the dissociation reaction is determined downstream after exit of the dissociation gas from the heating apparatus for the halogenated aliphatic hydrocarbon or at the top of the quenching column, preferably by means of an on-line analytical method, in particular by means of an on-line gas chromatograph.

61. An improved apparatus for the thermal dissociation of halogenated aliphatic hydrocarbons to form ethylenically unsaturated halogenated hydrocarbons therefrom, comprising:
a.) a process flow tube having sections for preheating, vaporizing, superheating and dissociating said aliphatic hydrocarbon;
b.) a reactor having at least one burner and having a convection zone and a radiation zone defined therein, at least a portion of the dissociating section of said process flow tube passing through said radiation zone and at least a portion of the preheating section being passing through said convection zone; and
c.) a heating apparatus for the halogenated aliphatic hydrocarbon which is located outside the reactor and is heated by the energy content of the reaction gases leaving the radiation zone,
wherein the improvement comprises:
i.) at least one means for initiating thermal dissociation of said halogenated aliphatic hydrocarbons, by introducing a free radical promoter chosen from the group consisting of chemical promoters, localized energy input sufficient to initiate formation of free radicals of chlorine or a combination thereof; each of said at least one means for initiating thermal dissociation being located along the process flow tube;
ii.) means for controlling the amount of free radical formation in the process flow tube such at least 50% of the halogenated aliphatic hydrocarbon used can be vaporized by the energy content of the reaction gases leaving the radiation zone without condensation of the reaction gases leaving the radiation zone; and
iii.) the heat exchange areas in the radiation zone, being dimensioned so that the average heat flux through the heat exchange area of the radiation zone is at least 35 kW/m².

62. The apparatus as claimed in claim 61, wherein the means of introducing chemical promoters for the thermal dissociation into the reaction tubes are feed lines which allow the introduction of predetermined amounts of chemical promoters into the feed gas stream.

63. The apparatus as claimed in claim 61, wherein the means of introducing chemical promoters for the thermal dissociation are feed lines which allow the introduction of predetermined amounts of chemical promoters into the reaction tubes at the level of the radiation zone, preferably feed lines which have nozzles at the reactor end, particularly preferably feed lines which open into the tubes in the first third, viewed in the flow direction of the reaction gas, of the radiation zone.

64. The apparatus as claimed in claim 61, wherein the means of introducing localized energy to promote the dissociation reaction in the reaction tubes at one or more points in the radiation zone are feed lines which preferably have nozzles at the reactor end and via which a thermal or nonthermal plasma is introduced into the reaction tubes at the level of the radiation zone or are windows via which electromagnetic radiation or particle beams are injected into the reaction tubes at the level of the radiation zone, particularly preferably feed lines or windows which open into or are installed in the tubes in the first third, viewed in the flow direction of the reaction gas, of the radiation zone.

65. The apparatus as claimed in claim 61, wherein the means of selecting the amount of the chemical promoter and/or the intensity of the localized energy input to form free radicals in the reaction tubes are regulating circuits in which a command variable is used to regulate the amount of the chemical promoter and/or the intensity of the localized energy input.

66. The apparatus as claimed in claim 61, wherein the temperature of the exiting reaction gases, the content of dissociation products in the reaction gases or the wall temperature of the reaction tubes at selected points is used as command variables.

67. The apparatus as claimed in claim 66, wherein the heating apparatus for the halogenated aliphatic hydrocarbon which is located outside the reactor comprises a first vessel and a second vessel, with the liquid halogenated aliphatic hydrocarbon being heated to boiling by the product gas in the first vessel and from there being transferred to the second vessel in which it is partly vaporized without further heating under a pressure which is lower than in the first vessel and the vaporized feed gas being fed into the reactor and the unvaporized halogenated aliphatic hydrocarbon being recirculated to the first vessel.

68. The apparatus as claimed in claim 61, wherein the heating apparatus for the halogenated aliphatic hydrocarbon which is located outside the reactor comprises a first vessel and a second vessel, with the liquid halogenated aliphatic hydrocarbon being heated to boiling by the product gas in the first vessel and from there being transferred to the second vessel in which it is partly vaporized without further heating under a pressure which is lower than in the first vessel and the vaporized feed gas being fed into the reactor and the unvaporized halogenated aliphatic hydrocarbon being recirculated to the first vessel.

69. The apparatus as claimed in claim 68, wherein the halogenated aliphatic hydrocarbon is conveyed in a pipe through the convection zone of the reactor where it is heated by means of the flue gas produced by the burners which heat the reactor before being fed into the second vessel.

70. The apparatus as claimed in claim 69, wherein at least one heat exchanger which is used for recovering waste heat from the condensation of the flue gas for preheating the combustion air or other media.
